(19) United States
(12) Patent Application Publication
(10) Pub. No.: US 2013/0274699 A1
(43) Pub. Date: Oct. 17, 2013

(54) PULL-ON DIAPER WITH OPEN BOTTOM AND OPTIONAL REMOVABLE FLAP

(71) Applicants: Chantelle D. Kelley, Lewisville, TX (US); Richard W. Borry, Lewisville, TX (US)

(72) Inventors: Chantelle D. Kelley, Lewisville, TX (US); Richard W. Borry, Lewisville, TX (US)

(21) Appl. No.: 13/825,761

(22) PCT Filed: Sep. 22, 2011
(86) PCT No.: PCT/US11/52729

§ 371 Date: Jul. 3, 2013

Related U.S. Application Data
(60) Provisional application No. 61/385,887, filed on Sep. 23, 2010.

(51) Int. Cl. A61F 13/496

(52) U.S. Cl. CPC A61F 13/496
USPC 604/385.09

(57) ABSTRACT

One embodiment relates to a diaper. The diaper includes a main body and an opening in the main body. The periphery of the opening is surrounded by the main body. The main body comprises two inwardly curved lateral edges. The opening is located centrally between the inwardly curved lateral edges. A flexible material is provided around the periphery of the opening. The opening is sized to leave the anus and genitals of the wearer exposed.

### CROSS-REFERENCE TO RELATED PATENT APPLICATIONS

The present application claims the benefit of U.S. Patent Application No. 61/385,887, filed Sep. 23, 2010 (Atty. Dkt. No. 079514-0105) which is incorporated herein by reference in its entirety.

### FIELD

The disclosure relates to the field of baby products. More particularly, the present disclosure relates to the field of diapers.

### BACKGROUND

The standard pull-on diaper can be difficult for young children to remove to sit on the toilet before they have already soiled their diaper. In addition, during toilet-training, young children sometimes have small wastes, which dirty only a small portion of the diaper but require replacement or cleaning of the entire diaper.

It would be advantageous to provide an improved diaper with an open portion and an optional cover over the open portion.

### SUMMARY

One embodiment relates to a diaper. The diaper includes a main body and an opening in the main body. The periphery of the opening is surrounded by the main body. The main body comprises two inwardly curved lateral edges. The opening is located centrally between the inwardly curved lateral edges. A flexible material is provided around the periphery of the opening. The opening is sized to leave the anus and genitals of the wearer exposed.

Another embodiment relates to a diaper assembly. The diaper assembly includes a diaper with a main body and an opening in the main body and an attached flap that covers the opening. The flap may be opened to allow a wearer to use a toilet without removing the diaper.

Still another embodiment relates to a diaper assembly. The diaper assembly includes a diaper with a main body and an opening in the main body and a removable flap that covers the opening. The flap may be removed to allow a wearer to use a toilet without removing the diaper. The flap further includes an absorbent portion to collect waste. A soiled flap may be replaced without needing to replace the diaper.

It is to be understood that both the foregoing general description and the following detailed description are exemplary and explanatory only, and are not restrictive of the invention as claimed.

### BRIEF DESCRIPTION OF THE DRAWINGS

These and other features, aspects, and advantages of the present invention will become apparent from the following description, appended claims, and the accompanying exemplary embodiments shown in the drawings, which are briefly described below.

FIG. 1 is a front perspective view of a pull-on diaper according to one embodiment.

FIG. 2 is a top view of a pull-on diaper of FIG. 1.

FIG. 3 is a top view of a pull-on diaper of FIG. 1, laid flat with the sides opened.

FIG. 4 is a rear perspective view of a pull-on diaper of FIG. 1.

FIG. 5 is a front perspective view of a pull-on diaper, according to another exemplary embodiment, with rear-affixed flap closed in front.

FIG. 6 is a rear perspective view of a pull-on diaper of FIG. 5, with flap open and affixed to the rear.

FIG. 7 is a front perspective view of a pull-on diaper according to another exemplary embodiment, when the cover is removed for replacement.

FIG. 8 is a top view of the cover from the pull-on diaper of FIG. 7, when the cover is removed for replacement.

FIG. 9 is a top view of the cover of FIG. 8 affixed to the inside of a pull-on diaper of FIG. 7, laid flat with the sides opened.

### DETAILED DESCRIPTION

In China, toddlers have worn “kaidangku” (literally, “open-crotch pants”) for decades (http://spankingartwiki.animeotk.com/wiki/Slit_pants). Recently, the Chinese government and urban culture have discouraged use of open-crotch pants in favor of diapers, due to concerns for public health (children squat and defecate wherever they are) and appearance in modern urban society. (http://www.chinadaily.com.cn/english/doc/2004-07/16/content_{—}349150.htm)

U.S. Pat. No. 4,944,733 (Casale, Jul. 31, 1990) and U.S. Pat. No. 5,843,065 (Wyant, Dec. 1, 1998) describe diapers with front openings, however, these work only for males and can be difficult in practice for young boys to operate. U.S. Pat. No. 5,217,447 (Gagnon, Jun. 8, 1993) describes a diaper system with detachable linings, however, the system requires an overgarment whose removal can be time-consuming when the wearer needs to relieve themselves.

According to an exemplary embodiment, an undergarment such as a diaper 10 includes an opening 12. The opening 12 (e.g., open portion, open section, etc.) is located along the centerline of diaper 10 between the leg openings 16. In various embodiments, opening 12 may be covered by a removable flap, panel, or other body. The removable body may therefore be replaced when soiled, reducing the need for the entire diaper to be removed.

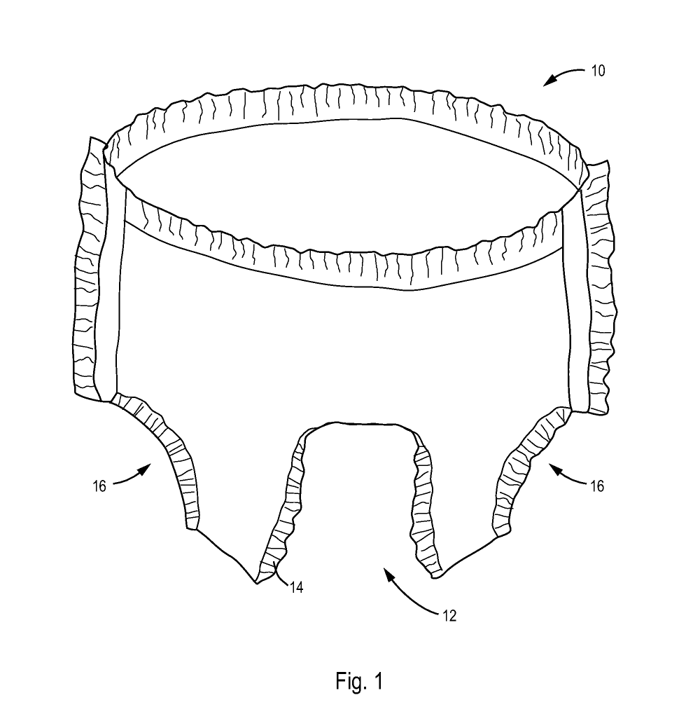

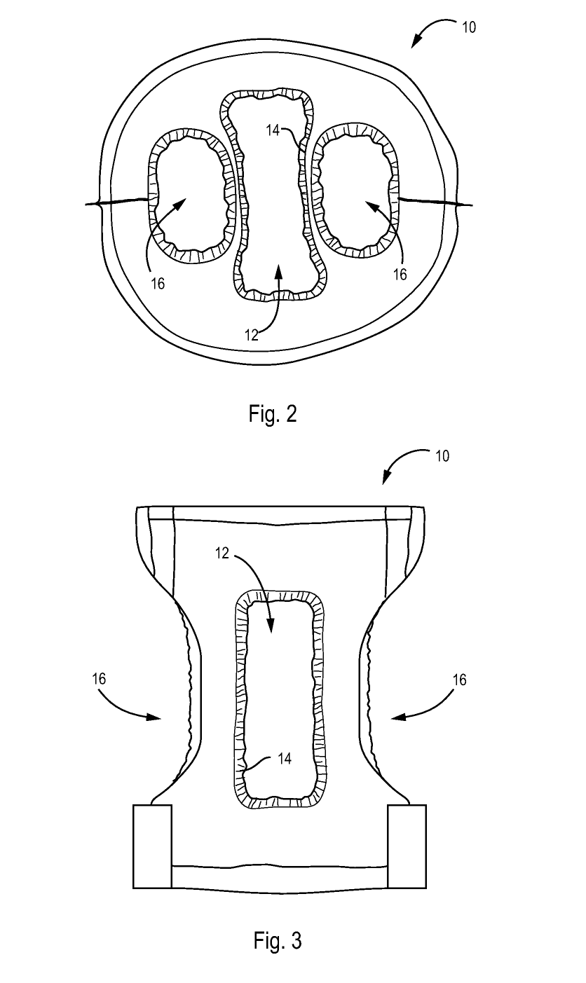

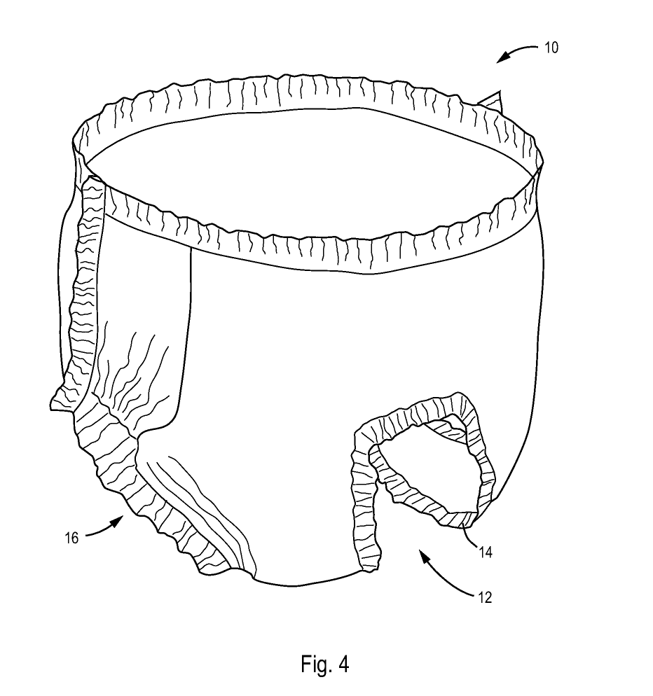

Referring now to the invention in its first embodiment, in FIG. 1 to FIG. 4 there is shown a pull-on diaper 10. FIG. 1 shows a front perspective view of the invention, FIG. 2 shows a top view and FIG. 4 a rear perspective view. FIG. 3 shows a top view of the pull-on diaper, with the sides pulled opened and diaper laid flat. The diaper 10 as shown in FIG. 1 to FIG. 4 may be made of cloth, plastic, or similar flexible fabric. The diaper 10 may be made of breathable materials to facilitate extended wear of the diaper 10.

Referring especially to FIG. 3, the diaper 10 includes an open section 12 in the crotch area located between two leg openings 16 formed by inwardly curved sides of the diaper. The opening 12 is sufficiently large to allow the wearer to sit on and use a training toilet without removing the pull-on diaper 10 and without soiling the material of the diaper 10.

A flexible material 14 (e.g., elastic, etc) lines the periphery of the open section 12. The flexible material 14 provides a snug fit to the wearer and to provide a feeling of comfort as if wearing a standard pull-on diaper.

In further detail, still referring to the invention of FIG. 1 to FIG. 4, the size and dimensions of the pull-on diaper 10 and opening 12 will vary depending on the size of the intended wearer, from infant to adult. The most common application, however, is expected to be young children of toilet-training age and size. The diaper 10 allows a young child to become accustomed to using a toilet while still maintaining the familiar feel of a traditional pull-on diaper. The opening 12 allows the toilet to be used even if the diaper 10 is not removed without soiling the diaper 10 and requiring the diaper 10 to be replaced.

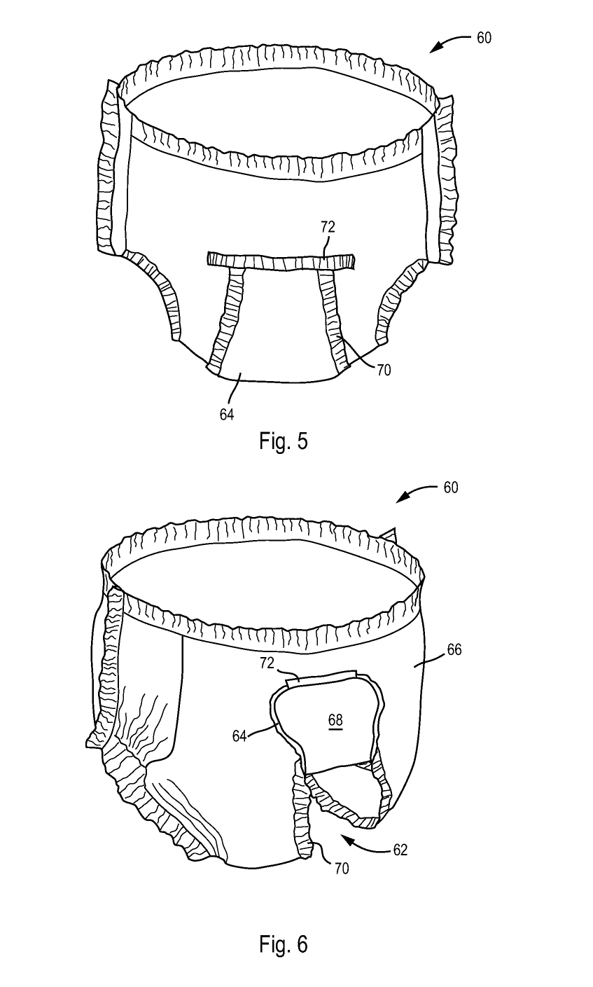

Referring now to FIG. 5 to FIG. 6, according to another exemplary embodiment, a pull-on diaper 60 includes an opening 62 in the crotch area and a flap 64 attached to the rear 66 of the opening 62. The flap 64 is lined on the inside with an absorbent material 68. The pull-on diaper 60 may be made of cloth, plastic, or similar flexible fabric. The diaper may be intended for disposal or wash and re-use. The diaper 60 may be made of breathable materials to facilitate extended wear of the diaper 60.

The opening 62 is sufficiently large to allow the wearer to sit on and use a training toilet without removing or soiling the pull-on diaper 60 when the flap 64 is open and affixed to the rear 66 of the pull-on diaper 60. Both the opening 62 and the flap 64 are lined with flexible material 70. The flexible material 70 provides a snug fit to the wearer and provides a feeling of comfort as if wearing a standard pull-on diaper. The flap 64 is configured to be relatively snug at the opening 62 and full in the middle to accumulate waste away from both the wearer and the diaper 60.

The flap 64 is moveable between an open position, wherein the opening 62 is unobstructed, and a closed position, wherein the flap 64 generally covers the opening 62. The flap 64 may include a tab 72 that is provided to affix the flap 64 to the diaper 60 in both the open position and the closed position. The flap design may also allow for sufficient adhesion without a tab 72 or strip of adhesive material on the flexible lining 70 along the openings.

Referring to FIG. 5, in a closed position, the tab 72 is affixed to the front of the diaper 60. When the flap 64 is in the closed position, the tab 72 is affixed to the front of the pull-on diaper 60 via a temporary closure (e.g., buttons, snaps, a hook and loop fastener, “loopless” technology, etc.). The flap 64 may also be affixed to the diaper 60 about the periphery of the opening 62. In a closed position, the flap 64 is sufficiently snug so as to prevent most liquid and solid waste from escaping the diaper 60.

The size and dimensions of the pull-on diaper 60, opening 62, and flap 64 will vary depending on the size of the intended wearer, from infant to adult.

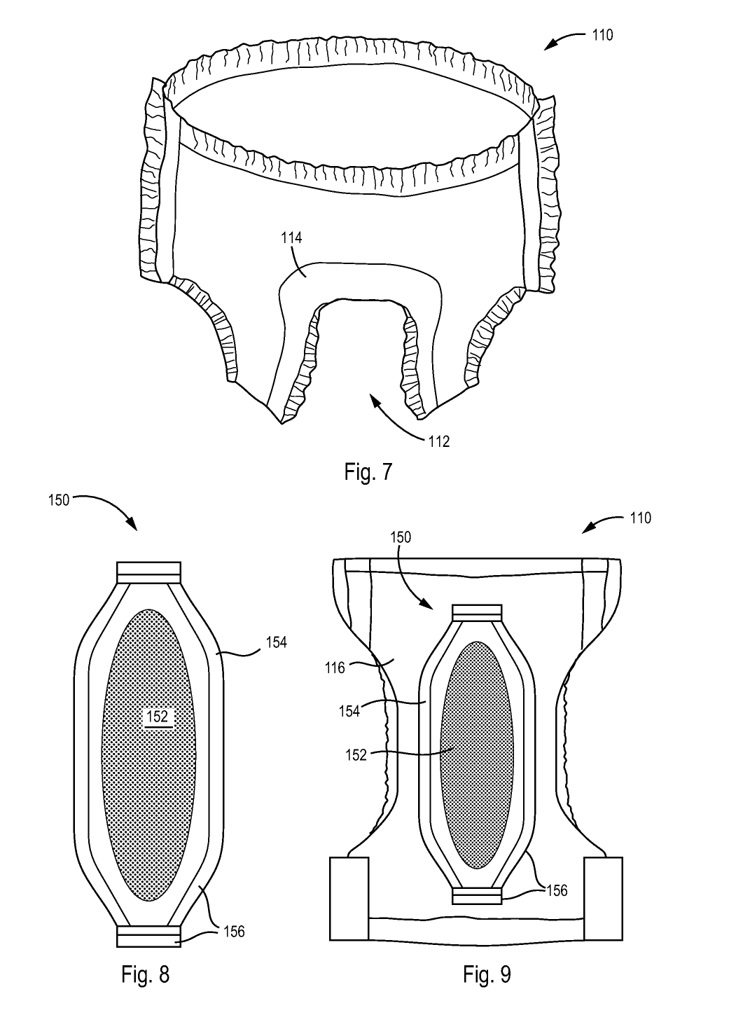

Referring now to FIG. 7 to FIG. 8, according to another exemplary embodiment, a pull-on diaper 110 includes an opening 112 in the crotch area and a removable cover 150. The removable cover 150 is affixed to at least a portion of the periphery of the opening 112 with a temporary adhesive material 114. The opening 112 is sufficiently large to allow the wearer to sit on and use a training toilet without removing the pull-on diaper 110 when the cover 150 is either removed or open without getting the diaper 110 dirty. The pull-on diaper 110 may be made of cloth, plastic, or similar flexible fabric. Both or either of the diaper and removable cover may be intended for disposal or wash and re-use. The diaper 110 and the cover 150 may be made of breathable materials to facilitate extended wear of the diaper 110. The diaper and cover design may also allow for sufficient adhesion without a separate strip of adhesive material 114, for example, using “loopless” or other technology. The cover 150 may be designed to affix to either the inside or outside of the diaper opening 112.

Shown in FIG. 8, the cover 150 is lined on the inside with an absorbent material 152, on the sides with a flexible material 154, and on the perimeter with a temporary adhesive material 156. Both the opening 112 and the cover 150 are lined with flexible material 154. Flexible material 154 provides a snug fit to the wearer and to provide a feeling of comfort as if wearing a standard pull-on diaper.

When the cover 150 is affixed over the opening 112, it is affixed to the front, sides, and rear via a temporary affixation method, and it is sufficiently snug so as to prevent most liquid and solid waste from escaping the diaper. The cover 150 can be removed completely and replaced, for example, when dirty. Or the cover 150 can be detached from the front and sides, but left attached to the rear, and folded back upon itself so that both ends of the cover 150 are affixed to the rear, for example, during toilet-training.

In one exemplary embodiment, cover 150 may be affixed to the outside of the diaper 110. In another exemplary embodiment, as shown in FIG. 9, cover 150 may be affixed to the inner surface 116 of the diaper 110.

According to one exemplary embodiment, the temporary adhesive material 156 coupling the cover 150 to the diaper 150 is a hook and loop fastener. According to other exemplary embodiments, the cover 150 may be coupled to the diaper 150 with any other suitable temporary coupling method, such as “loopless” technology, an adhesive, buttons, snaps, etc.

The absorbency and size of the cover 150 is designed to be sufficient to catch most liquid and small solid wastes, thus allowing the cover 150 to be replaced without replacing the entire diaper 110 on the wearer. The cover is configured to be relatively snug at the opening 112 and full in the middle to accumulate waste away from both the wearer and the diaper 110. Large solid wastes are expected to require replacement of both the cover 150 and pull-on diaper 110. The size and dimensions of the pull-on diaper 110, opening 112, and cover 150 will vary depending on the size of the intended wearer, from infant to adult.

According to an exemplary embodiment, the diaper (e.g., diaper 10, diaper 60, or diaper 110, etc.) may be formed of a hydrophobic (water-repellant) material and the replaceable portion (e.g., flap 64 or cover 150) may be formed of a hydrophilic (water-loving) material. In use, the diaper may be worn for hours with only replacement of the flap or cover; thus, for comfort of the wearer, the diaper material may be a breathable fabric that reduces skin irritation and prevents accumulation of sweat within the diaper. The material choice and shape of the replaceable portion is intended to reduce diaper waste by keeping the diaper clean and dry, and concentrating all liquid and small solid waste into the replaceable portion.

The advantages of the diaper with an opening and an optional cover as described in several exemplary embodiments above include, without limitation, the ability to toilet-train the wearer by providing the feeling of comfort of wearing a pull-on diaper, while being able to quickly sit the wearer on a toilet when they need to use it. In the second and third embodiment, the wearer or care-giver can simply open the flap or cover when the wearer needs to use the toilet, without having to pull the diaper down. In the third embodiment, the cover may be removed and replaced without replacing the pull-on diaper, if the wearer has a small waste prior to being able to sit on a toilet, so that only the cover is dirty. If the diaper is made of disposable materials, then this option allows for significantly less material disposed compared to replacing the entire pull-on diaper. Providing a removable, disposable cover may reduce the number of changes required for the entire diaper assembly will be required, which may reduce the lower total daily cost of use to the wearer or their caregiver.

In broad embodiment, the present invention is an improved pull-on diaper that has a variety of opening options to allow easier access to the wearer's bottom, for toilet-training or general care. Although the most common application is expected to be young children of toilet-training age, the invention may also apply to infants or adults, or to other animals such as dogs and cats. The size, dimensions, and other modifications will adjust depending on the wearer, for example, the rear of the pull-on diaper may have an additional hole for a tail when used for certain animals.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. For example, although different example embodiments may have been described as including one or more features providing one or more benefits, it is contemplated that the described features may be interchanged with one another or alternatively be combined with one another in the described example embodiments or in other alternative embodiments. Because the technology of the present disclosure is relatively complex, not all changes in the technology are foreseeable. The present disclosure described with reference to the example is manifestly intended to be as broad as possible. For example, unless specifically otherwise noted a single particular element may also encompass a plurality of such particular elements. The invention should therefore not be limited by the above described embodiment, method, and examples, but by all embodiments and methods within the scope and spirit of the invention.

It is also important to note that the construction and arrangement of the elements of the system as shown in the exemplary embodiments is illustrative only. Although only a certain number of embodiments have been described in detail in this disclosure, those skilled in the art who review this disclosure will readily appreciate that many modifications are possible (e.g., variations in sizes, dimensions, structures, shapes and proportions of the various elements, values of parameters, mounting arrangements, use of materials, colors, orientations, etc.) without materially departing from the novel teachings and advantages of the subject matter recited.

Further, elements shown as integrally formed may be constructed of multiple parts or elements shown as multiple parts may be integrally formed, the operation of the assemblies may be reversed or otherwise varied, the length or width of the structures and/or members or connectors or other elements of the system may be varied, the nature or number of adjustment or attachment positions provided between the elements may be varied. It should be noted that the elements and/or assemblies of the system may be constructed from any of a wide variety of materials that provide sufficient strength or durability. Accordingly, all such modifications are intended to be included within the scope of the present disclosure. Other substitutions, modifications, changes and omissions may be made in the design, operating conditions and arrangement of the exemplary embodiments without departing from the spirit of the present subject matter.

What is claimed is:

1. A diaper comprising:
a main body comprising an opening, and
two lateral edges, wherein
the periphery of the opening is surrounded by the main body and the opening is located centrally between the lateral edges.

2. The diaper claimed in claim 1, wherein a flexible material is provided around the periphery of the opening.

3. The diaper claimed in claim 1, wherein, the opening is sized to leave the anus and genitals of the wearer exposed.

4. A diaper assembly comprising:
a diaper, as claimed in claim 1, and
an attached flap that covers the opening, wherein
the flap may be opened to allow a wearer to use a toilet without removing the diaper.

5. The diaper assembly claimed in claim 4, wherein the flap includes an absorbent portion to collect waste.

6. A diaper assembly comprising:
a diaper, as claimed in claim 1, and
a removable flap that covers the opening, wherein
the flap may be removed to allow a wearer to use a toilet without removing the diaper.

7. The diaper assembly claimed in claim 6, wherein the flap includes an absorbent portion to collect waste
